(19) United States
(12) Patent Application Publication
(10) Pub. No.: US 2016/0130201 A1
(43) Pub. Date: May 12, 2016

(54) PROCESSES FOR SELECTIVE EXTRACTION OF UNSAPONIFIABLE MATERIALS FROM RENEWABLE RAW MATERIALS BY LIQUID-LIQUID EXTRACTION IN THE PRESENCE OF A COSOLVENT

(71) Applicant: SAEML VALAGRO CARBONE RENOUVELABLE POITOU- CHARENTES, Poitiers Cedex (FR)

(72) Inventor: Antoine PICCIRILLI, Poitiers (FR)

(21) Appl. No.: 14/896,177

(22) PCT Filed: Jun. 4, 2014
(86) PCT No.: PCT/FR2014/051329

§ 371 Date: Dec. 4, 2015

(30) Foreign Application Priority Data
Jun. 4, 2013 (FR) 1355141

(51) Int. Cl. C07C 29/86; C07D 307/36

(52) U.S. Cl. CPC C07C 29/86; C07D 307/36

(57) ABSTRACT

A method for extracting an unsaponifiable fraction from a solid renewable raw material, includes the extraction of the fats from the solid renewable raw material, leading to the production of an oil, the concentration of the oil so as to obtain a mixture enriched in unsaponifiable fraction, and the liquid-liquid extraction of the mixture enriched in unsaponifiable fraction, in the presence of at least one polar organic solvent and at least one non-polar cosolvent immiscible with the polar organic solvent, resulting in the formation of an organic polar phase enriched in lipids functionalized with one or more function(s) chosen from hydroxyl, epoxide, ketone, thiol, aldehyde, ether and amine functions, and to the formation of a non-polar organic phase enriched in lipids containing no or few hydroxyl, epoxide, ketone, thiol, aldehyde, ether and amine function(s), then the concentration of the organic phases.

The present invention relates to the oleochemical field. More particularly, this invention relates to a method for extracting unsaponifiable matters from a lipidic renewable raw material, especially from an oleiferous fruit, in particular avocado, from an oleaginous seed or from a raw material derived from animals, algae, fungi or yeasts, or from a microorganism.

As used herein, lipids are intended to mean substances of biological origin that are soluble in non-polar solvents. Lipids may be saponifiable (for example triglycerides) or not saponifiable (for example molecules structured with a steroid-type skeleton).

As used herein, unsaponifiable matters are intended to include all the compounds, which, after complete saponification of a fat, that is to say under the sustained action of an alkaline base, remain insoluble in water and may be extracted by an organic solvent in which they are soluble. The unsaponifiable matters generally represent a minor fraction in the fat.

There are five major groups of substances in most of unsaponifiable matters derived from vegetable fats: saturated or unsaturated hydrocarbons, aliphatic or terpene alcohols, sterols, tocopherols and tocotrienols, and carotenoid pigments, especially xanthophylls.

Lipidic renewable raw materials comprise highly variable proportions of unsaponifiable compounds. The unsaponifiable fraction contents obtained by extracting various vegetable oils according to different known methods range from 1 to 7% by weight of unsaponifiable matters in avocado oil, as opposed to 0.5% in coconut oil and 1% in soya or olive oil.

Currently, the traditional methods for extracting unsaponifiable matters generally use as a lipidic raw material vegetable oils and derivatives thereof and co-products from the lipid extraction industry (vegetable oils, animal fats, marine fats and oils, vegetable oleoresins), resulting from their refining and processing. Most of the time, it is necessary to extract the unsaponifiable matters from raw, semi-refined or refined vegetable oils, from unsaponifiable matter concentrates derived from refined oils obtained through a molecular distillation or through an extraction using supercritical fluids. Also, a number of unsaponifiable fractions such as sterols, squalene, tocopherols or tocotrienols are obtained from the vegetable oils from deodorization emissions, which are abundant co-products resulting from the chemical or physical refining of vegetable oils. However, to be mentioned as other co-products resulting from the refining of lipids are also acid-containing oils, soap pastes, lipids retained by bleaching earths that are used for decolorizing oils, earths retrieved from winterization units. Moreover, co-products resulting from oilseed or oleiferous fruit grinding may also be used, such as oil-cakes, seed husks or stones, molasses, black liquors.

In order to extract unsaponifiable matters or fractions thereof, co-products from the processing of lipids may also be used, such as raw glycerins from biodiesel production plants, resulting from animal or vegetable fat hydrolysis or saponification processes, greasy waters from animal fat processing industries, fatty acid alkyl ester still bottoms.

Likewise, unsaponifiable fractions are produced, especially sterols, from industrial co-products such as pulp productions called tall oil. Also to be mentioned are unsaponifiable fractions of co-products resulting from the extraction process of beverages, such as industrial breweries, rum distilleries, and malting plants.

As a raw material, source for unsaponifiable matters, can be further employed plant serums (ex. from tomatoes, citrus fruits), seeds, integuments, oleoresins from fruits that are oleiferous or not, vegetables, flowers or leaves.

The methods for extracting unsaponifiable matters most of the time comprise a step of transesterification or esterification of the fat obtained by pressing, and/or a step of saponification of the fat, followed with a liquid-liquid extraction by means of an organic solvent.

The methods for selectively extracting unsaponifiable fractions are not numerous.

The application WO 2011/048339 describes a method for extracting an unsaponifiable fraction from a renewable raw material, comprising a) the dehydration and conditioning of the renewable raw material, b) the transesterification by an active trituration of the conditioned lipid raw material in the presence of a light alcohol and a catalyst, c) the evaporation of the light alcohol, d) the concentration of the liquid phase so as to obtain a concentrate comprising the unsaponifiable fraction diluted in fatty acid alkyl esters, e) the saponification of the unsaponifiable concentrate, f) the extraction of the unsaponifiable fraction from the saponified mixture.

Avocado, because of its unsaponifiable fraction high content should be considered with a very special attention. It allows in a known way the access to particular lipids of the furanic type, which major component is a linoleic furan noted H7 having the following formula:

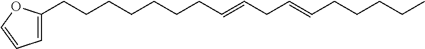 [SMILES: CCCCC/C=C/C/C=C/CCCCCCCc1ccco1]

As used herein, avocado-derived furan lipids are intended to mean components having the following formula:

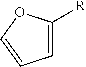

wherein R is a C11-C19, preferably a C13-C17, linear hydrocarbon chain, saturated or comprising one or more ethylene or acetylene unsaturations. These furan lipids from avocado have been described especially in Farines, M. and al, 1995, J. Am. Oil Chem. Soc. 72, 473. As a rule, furan lipids from avocado are compounds that are unique in the vegetable kingdom and are very particularly sought after for their pharmacological, cosmetic, and nutritional properties, or even as biopesticides.

Furan lipids from avocado are metabolites of precursor compounds that are initially present in the fruit and the leaves, and which, due to the effect of heat do dehydrate and cyclize to furan derivatives. As an example, linoleic furan H7 results from the heat transformation of following keto-hydroxyl precursor, noted P1 H7:

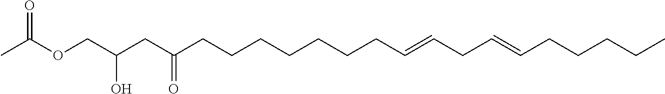 [SMILES: CCCCC/C=C/C/C=C/CCCCCCCC(=O)CC(O)COC(C)=O]

Under atmospheric pressure, precursor P1 H7 is typically converted to linoleic furan H7 at a temperature ranging from 80 to 120° C.

It is today well established that the presence of these furanic compound precursors in the leaves or in the fruit of avocado (including the stone) not only depends on the variety (Hass and Fuerte varieties being the richest in such compounds) but also on the method for producing the oil or other vegetable extract of avocado (hexane or ethanol extract from avocado leaves).

Furthermore, some compounds that are initially present in avocado fruit and leaves may present in the form of polyhydroxylated fatty alcohols, most of the time non acetylated, such as the following compound:

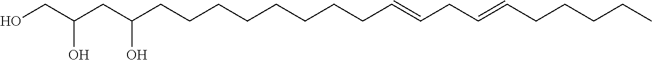 [SMILES: CCCCC/C=C/C/C=C/CCCCCCCCC(O)CC(O)CO]

As used herein, a polyhydroxylated fatty alcohol from avocado is intended to mean a polyol in the form of a C17-C21 straight main hydrocarbon chain, saturated or comprising one or more ethylene or acetylene unsaturations, and comprising at least two hydroxyl groups, said hydroxyl groups being generally located on one portion of the main chain, preferably in the direction of either of both ends thereof, the other portion of this main chain thus forming the fatty chain (hydrophobic portion) of the polyol.

The polyhydroxylated fatty alcohol content in the fruit mainly depends on the weather conditions, on the soil quality, on the season and on the ripening of the fruits when picked.

Considering the therapeutic interest of the avocado unsaponifiable, that is rich in furan lipids, for its beneficial and curative effect onto conjunctive tissues, especially against inflammatory diseases such as arthrosis, parodontitis and scleroderma, and further considering its generally high cost, there is a strong need for preparing with the best yield as possible, unsaponifiable fractions from avocado oil, that would be rich in furan lipids. Likewise, there is a real interest in positively using, with a maximum yield, the fruit as a whole, so as to improve the global cost effectiveness of the process.

The known methods to produce these furanic compounds or specific polyols from the fruit or from the oil extracted from the fruit avocado do only enable to obtain these compounds when combined with many other avocado-derived unsaponifiable compounds.

The French application FR 2678632 describes a method for producing the avocado unsaponifiable fraction from an avocado oil enriched with one of its fractions, called the H fraction, in fact corresponding to the same furan lipids. The preparation of such a furan lipid-rich unsaponifiable matter, which content may vary from 30 to 60%, essentially depends on the controlled heating of the fresh fruits, that have been beforehand thinly sliced, at a temperature ranging from 80 to 120° C., and for a period of time preferably chosen between 24 and 48 hours. This heat treatment enables after extraction, to obtain a furan lipid-rich avocado oil. Lastly, starting from this oil, the unsaponifiable fraction is obtained according to a traditional saponification method, completed with a step of liquid-liquid extraction using an organic solvent.

The application WO 01/21605 describes a method for extracting furan lipid compounds and polyhydroxylated fatty alcohols from avocado, comprising a heat treatment of the fruit at a temperature of at least 80° C. (controlled drying), the extraction of oil by cold pressing, the enrichment with unsaponifiable matter through cold crystallization or liquid-liquid extraction or molecular distillation, the ethanolic potash-mediated saponification, the unsaponifiable extraction in counter-current column with an organic solvent, followed with steps of filtration, washing, solvent removing, deodorization and final molecular distillation. This method makes it possible to obtain either a distillate comprising primarily avocado furan lipids, or a distillate comprising primarily avocado furan lipids and polyhydroxylated fatty alcohols. However such method only enables to take advantage of a minor part of the fruit.

Indeed, in this type of process, the oil forming the bottoms resulting from the step of concentration of the unsaponifiable matter by molecular distillation, i.e. around 90% of the oil extracted from the fruit, can hardly be positively reused. This strongly colored oil did indeed undergo a heat treatment through high temperature-distillation, which leads to an automatic and non-reversible destruction of the chlorophyllous pigments, as well as phospholipids, with a very detrimental effect on the future refining of the distilled crude oil. Only a highly advanced refining of this oil, in the best case scenario, enables to give a relatively acceptable color back to it. Refining requires a high consumption of inputs (such as bleaching earths), of energy and still remains very brutal for unsaturated fatty acids (isomerization). Lastly, an exogenous antioxidant must be added for the preservation of this refined oil for a commercially acceptable period of time. As a consequence, the thus refined oil can absolutely not be reused for human nutrition or in specialist pharmaceutical applications.

A further drawback of this method consists in the production of an oil cake unsuitable for animal feeding. The latter indeed contains antinutritional compounds (toxic H precursors, used as biopesticides, furan lipids) and proteins that have been highly degraded during the extraction by mechanical pressing of the air-dried fruits (de facto highly oxidized), which suffer from a very low digestibility. As a consequence, the oil cake or proteins thereof, cannot be used in animal feeding and even less in human nutrition, even if the flesh of the fruit is commonly consumed by humans (guacamole, fruit to be directly consumed).

In the same way, the noble polysaccharides within the fruit, such as perseitol and nanoheptulose, unique sugars in the vegetable kingdom, with demonstrated pharmaceutical, cosmetic and nutritional properties (for ex. improved liver function), are partially destroyed through a Maillard reaction and/or caramelization process induced by the mechanical pressure of the dehydrated fruits, or are made very difficult to extract because of the excessive interaction with the fiber and protein-containing matrix.

As a conclusion, this type of method only enables a poor reuse of the fruit, which can be estimated to be lower than 15%.

As a consequence, it remains necessary to improve the yield as well as the selectivity of the methods for extracting furan lipids and/or polyhydroxylated fatty alcohols from avocado.

There is thus still a need for a method for selectively extracting unsaponifiable matters from fats while preserving the fruit integrity for a better future reuse, which implementation would be economic and would make it possible to also recover co-products of glycerides with a higher added value than free fatty acids, or proteins and polysaccharides with a good nutritional quality. It further would be desirable to develop a method for high-yield extracting unsaponifiable matters relative to the polarity of their fractions. It is indeed desirable to provide a robust method to selectively produce the expected fractions without being detrimental to the other interesting fractions or parts of the fruit.

In response, it is an object of the present invention to provide a method for extracting an unsaponifiable fraction from a solid renewable raw material comprising fats, and especially lipids functionalized with one or more function(s) chosen from hydroxyl, epoxide, ketone, thiol, aldehyde, ether and amine functions, comprising the following steps:

a) optional dehydration, possibly preceded or followed with a conditioning of the renewable raw material,

b) extraction of the fats from the raw material optionally dehydrated and optionally conditioned to obtain an oil,

c) concentration of the oil resulting from step b) so as to obtain a mixture enriched with the unsaponifiable fraction,

d) liquid-liquid extraction of the mixture enriched with the unsaponifiable fraction in the presence of at least one polar organic solvent and at least one non-polar cosolvent immiscible with said polar organic solvent, resulting in the formation of a polar organic phase enriched with lipids functionalized with one or more function(s) chosen from hydroxyl, epoxide, ketone, thiol, aldehyde, ether and amine functions, and optionally comprising the following steps:

e) saponification of the polar organic phase, optionally preceded, accompanied or followed with a heat treatment at a temperature higher than or equal to 75° C., preferably higher than or equal to 80° C.,

f) extraction of the unsaponifiable fraction from the saponified mixture.

The present invention further relates to a method for extracting an unsaponifiable fraction from a solid renewable fat-containing raw material, comprising the following steps:

a) optional dehydration possibly preceded or followed with a conditioning of the renewable raw material,

b) extraction of the fats from the raw material optionally dehydrated and optionally conditioned to obtain an oil,

c) concentration of the oil resulting from step b) so as to obtain a mixture enriched with the unsaponifiable fraction,

d) liquid-liquid extraction of the mixture enriched with the unsaponifiable fraction in the presence of at least one polar organic solvent and at least one non-polar cosolvent immiscible with said polar organic solvent, resulting in the formation of a non-polar organic phase enriched with lipids containing no or few hydroxyl, epoxide, ketone, thiol, aldehyde, ether and amine function(s),

and optionally comprising the following steps:

e) saponification of the non-polar organic phase,

f) extraction of the unsaponifiable fraction from the saponified mixture,

wherein said renewable raw material undergoes optionally a heat treatment at a temperature higher than or equal to 75° C., preferably higher than or equal to 80° C., before step d).

Both methods of the invention do differ in that the first method aims at recovering an unsaponifiable fraction soluble in a polar phase (or which precursors are soluble in such a phase), whereas the second method aims at recovering the unsaponifiable fraction soluble in a non-polar organic phase (or which metabolites are soluble in such a phase). In the case of an avocado, both methods, although different in numerous steps, are however both equally useful since they make it possible to selectively recover furan lipids from the unsaponifiable fraction with a high yield, while enabling the production of very high quality-coproducts, which can be positively reused: distilled alkyl esters of avocado oil, perfectly traced avocado glycerin, oil cakes with antinutritional compounds removed therefrom, which can be potentially used as sources of proteins, of oligopeptides, of perseitol and nanoheptulose, avocado fibers.

In the particular case of avocado, the raw materials in the first method especially are not initially heated at a high temperature (they are only heated after the liquid-liquid extraction step), while they are heated before the liquid-liquid extraction step in the second method, so as to produce earlier the furanic compound characteristics of a thermally treated avocado. In the case of the first method, the liquid-liquid extraction step is implemented with avocados, which did not undergo such a heat treatment and thus, at this stage, do contain furan lipid precursors.

The present invention therefore aims at extracting an unsaponifiable fraction from a renewable lipid raw material in a solid form, generally originating from a plant or an animal, preferably from a plant. This raw material may especially be chosen from oleiferous fruits, oleaginous seeds, oleoproteaginous seeds, seed hulls, oleaginous almonds, sprouts, fruit stones and cuticles, raw materials derived from animals, algae, fungi or yeasts, or from a microorganism, and that are rich in lipids.

In a first embodiment, the implemented solid raw material is an oleiferous fruit, which may be, without limitation, olive, shea, amaranth, palm, buritti, tucuman, squash, Serenoa repens, African palm or avocado.

In a second embodiment, the solid raw material is a seed, a pit, a sprout, a cuticle or a stone from a vegetable raw material chosen from rapeseed, soybean, sunflower, cotton, wheat, corn, rice, grapes (seeds), walnut, hazelnut, jojoba, lupine, camelina, flax, coconut, safflower, crambe, copra, peanuts, jatropha, castor bean, neem, canker, Cuphea, lesquerella, Inca inchi, perilla, echium, evening primrose, borage, black currant, pine of Korea, China wood, cotton, poppy (seeds), sesame, amaranth, coffee, oats, tomatoes, mastic tree, marigold, karanja, rice bran, Brazil nuts, andiroba, schizandra, ucuhuba, cupuacu, murumuru, pequi, seeds from lemon oil, mandarin, orange, watermelon, Cucurbita pepo and tomato. The lipid raw material may also be a raw material derived from animals, algae, fungi or yeasts. To be mentioned as preferred animal raw materials are fish liver and skin, very especially those of shark, cod and chimera, as well as solid waste from the meat industry (brains, tendons, lanolin . . . ).

Other vegetable raw materials containing oleoresins that are rich in unsaponifiable matters are tomato, marigold, paprika, rosemary.

To be mentioned as suitable examples of algae containing interesting unsaponifiable compounds are microalgae Duniella salina (rich in beta-carotene) and Hematococcus pluvialis (rich in asthaxanthin). Suitable examples of microorganisms, especially bacteria containing interesting unsaponifiable compounds include any mycelia or other mold and fungus (production of ergosterol), Phaffia sp. (producing asthaxanthin), Blakeslea trispora, (producing lycopene and phytoene), Muriellopsis sp. (producing lutein), or are especially mentioned in the application WO 2012/159980 (microalgae strain adapted to produce squalene), in the American patent U.S. Pat. No. 7,659,097 (bacteria producing especially farnesol and farnesene), in the publication Pure & Appl. Chem., Vol. 69, No. 10, pp. 2169-2173,1997 (production of carotenoids) or in Journal of Biomedicine and Biotechnology, 2012;2012:607329, doi: 10.1155/2012/607329 (biotechnological production of co-enzyme Q10).

It is desirable that the raw materials used in the method of the invention have an acidity lower than 3 mg KOH/g. Indeed, higher contents in free fatty acids in these raw materials would cause the formation of soaps in a basic medium. As used herein, fatty acids are intended to mean C4-C28 mono-, di- or tricarboxylic aliphatic acids, saturated, monounsaturated or polyunsaturated, linear or branched, cyclic or acyclic, that may comprise some particular organic functions (hydroxyl, epoxy functions, . . . ).

The first method of the invention will now be presented in detail.

The raw materials that are implemented in the first method of the invention comprise lipid components functionalized with one or more polar function(s), chosen from (preferably aliphatic) hydroxyl, epoxide, ketone, thiol, aldehyde, ether and amine functions, as for example avocado, karanja, jatropha, andiroba, neem, schizandra, lupine hull, cashew nut, sesame, rice bran, cotton, or oil-producing raw materials that are rich in phytosterols such as corn, soya, sunflower, rapeseed, which all are very rich in such compounds.

This method comprises optionally a first step a) of dehydration and/or of conditioning of the renewable raw material. Dehydration and conditioning, when conducted at a temperature lower than or equal to 80° C., preferably lower than or equal to 75° C., are said to be controlled (this is required for avocado). Said temperature is preferably higher than or equal to −50° C. According to another embodiment (not applicable to avocado), temperature varies from 50 to 120° C., more preferably from 75 to 120° C. Dehydration may be conducted under inert atmosphere, especially in the case of raw materials containing delicate compounds that may oxidize when temperature increases. It is preferably conducted under atmospheric pressure.

In the case of avocado (which is intended to mean, as used in the present application, the fruit, the stone, the leaves of avocado or their mixtures), not to rise temperature above 75 or 80° C. prevents the conversion of furan lipid precursors to furan lipids.

Dehydration may be implemented before or after conditioning (if needed). Preferably, oleiferous fruits like avocado are dehydrated prior to being conditioned, whereas oleaginous seeds on the contrary are first conditioned prior to being dehydrated.

As used herein, dehydration is intended to include all the techniques known from the person skilled in the art, which enable the total or partial removal of water from the raw material. Amongst these techniques are to be mentioned, without limitation, fluidized bed drying, drying under a hot air current or under an inert atmosphere (ex. nitrogen), packed-bed drying, under atmospheric pressure or under vacuum, thick-layer drying or thin-layer drying, in a continuous belt dryer in a hot air dryer with rotary fans, or microwave drying, spray drying, freeze-drying and osmotic dehydration, in a solution (direct osmosis), or in a solid phase (ex. drying in osmotic bags), drying using solid absorbents, such as zeolites or molecular sieves.

More preferably, the drying time and temperature are chosen so that residual moisture is lower than or equal to 10% by weight, preferably lower than or equal to 3% by weight, more preferably lower than or equal to 2%, as compared to the weight of the lipid raw material obtained at the end of the dehydration step. The residual moisture of the raw material may be determined by thermogravimetry. This drying step will make the lipid component extraction more efficient, because it especially makes the cells of the raw material burst, and the oil-in-water emulsion break, such as present in this raw material. Moreover it may facilitate the conditioning of the raw material, especially the crushing or milling operations, which will make the solvent-mediated extraction more efficient because of the benefit in terms of contact surface with the solvents.

Within the frame of the present method, so as to facilitate an industrial implementation and for cost reasons, drying in thermoregulated, vented dryers (drying ovens), in thin layers and under a hot air current, is preferred. The temperature does preferably range from 70 to 75° C., and dehydration lasts preferably for 8 to 36 hours.

The aim of the optional conditioning of the raw material is to make the fats the most accessible to the extraction solvents and to catalysts, especially through a simple phenomenon of percolation. Conditioning may also increase the specific surface and porosity of the raw material in contact with these reagents. The conditioning of the raw material does not lead to any fat extraction.

Preferably, the renewable raw material is conditioned by flattening, flocking, blowing or grinding in the form of a powder. As an example, the raw material may be toasted or flocked, or conditioned and/or freeze-dried, dried through evaporation, spraying, mechanical grinding, freeze-grinding, dehulling, flash-relaxation (quick drying by creation of vacuum and quick depressurization), conditioned with pulsed electromagnetic fields, by reactive or non-reactive extrusion, flattening by means of a mechanical flattener with smooth rollers or corrugated rollers, blowing through hot air or superheated vapor supply. In the case of avocado, primarily cut avocado fruits will be used, which will be thereafter submitted to a controlled dehydration step, and lastly the dried fruit will be conditioned, generally by grinding the fresh pulp.

The solid renewable raw material optionally dehydrated and/or conditioned is submitted to a step b) for extracting the fats thereof leading to the production of an oil. This step is preferably performed in the absence of catalyst, especially with no basic catalyst.

Step b) is conducted under temperature and duration conditions sufficient to enable the extraction of fats, that is to say of triglycerides and other lipid components from the solid raw material, leading to the formation of an oil cake and of a mixture comprising unsaponifiable compounds and saponifiable compounds, especially triglycerides, as well as, depending on the type of raw material used, soluble polysaccharides, phenolic compounds, glucosinolates, isocyanates, polar alkaloids, polar terpenes.

Step b) however is conducted at a temperature lower than or equal to 80° C., preferably lower than or equal to 75° C. in the case of avocado especially, such temperature control preventing furan lipid precursors to be converted to furan lipids. These remain present in their hydroxylated form (not cyclized to furans) during the fruit extraction.

In other cases, step b) may be conducted without limitation as regards temperature, that is to say the temperature may be set over 75 or 80° C. Thus, when the raw material is not derived from avocado, step b) may be conducted by implementing a heating process at a temperature ranging from 40 to 100° C. Step b) generally is conducted at room temperature but may also be conducted by implementing a heating process, at a temperature preferably of at least 40° C. and preferably lower than or equal to 80° C., preferably lower than or equal to 75° C.

This oil extraction step may especially imply one or more pressing and/or centrifugation operations, so as to extract fats as an oil from the solid renewable raw material. This transformation step is a traditional process perfectly mastered by the person skilled in the art. The most preferred extraction mode is a mechanical pressing, which enables to isolate the oil from an oil cake, especially a cold pressing or a pressing including a heating process, wherein the mechanical pressing may be effected for example in a screw press or in a hydraulic press. The extraction may also be carried out by putting the solid raw material in contact with a suitable organic solvent, for example hexane, methanol or a methanol-chloroform combination, this solvent or another solvent can also be used for washing the oil cake. In this case, oil is recovered after evaporation of the solvent, in particular under reduced pressure, while making sure when a heating process is provided during evaporation that the temperature remains lower than or equal to 80° C., preferably lower than or equal to 75° C. in the case of an avocado, so as to prevent the conversion of furan lipid precursors to furan lipids. Extraction methods by pressing and using a solvent may also be combined, for example by submitting the oil cake resulting from a mechanical pressing to a solvent-mediated extraction.

The oil cake, containing solvent or not, may be dried, then be directly used especially in animal feeding.

Prior to conducting the following step, the oil extract may be submitted to a filtration step.

The resulting lipid phase may optionally be submitted to a transesterification step in the presence of at least one polar organic solvent comprising at least one light alcohol such as defined hereunder and at least one catalyst, before or after concentration step c), preferably before. In any event, the transesterification must be carried out before step e) of saponification.

This optional step converts glycerides to fatty acid esters and releases glycerol in the case of triglycerides. Preferably a monoalcohol is used, which generates fatty acid monoesters, more preferably an alkyl monoalcohol, which generates fatty acid alkyl monoesters. The transesterification should be carried out as regards temperature with the same safe practice as in step b).

The catalyst is preferably a basic catalyst preferably chosen from alcoholic soda, solid soda, alcoholic potash, solid potash, alkaline alcoholates, such as lithium, sodium or potassium methylate, ethylate, n-propylate, isopropylate, n-butylate, i-butylate or t-butylate, amines and polyamines, or an acid catalyst preferably chosen from sulfuric acid, nitric acid, paratoluenesulfonic acid, hydrochloric acid and Lewis acids. An acid catalyst will be more particularly used in extreme situations, where free acidity of the fat will be higher than 4 mg KOH/g. This step will lead to the esterification of free fatty acids, and the continuation of the method consists in continuing with a base-catalyzed transesterification reaction.

The transesterification step may be conducted especially in a batch reactor with a stirred bed or in a continuous reactor with a mobile belt, of the continuous extractor type. In a preferred embodiment, the organic solvent and the organic oil resulting from step b) are introduced in counter-current to each other into a reactor. To optimize the conversion of the mono-, di- and triglycerides to fatty acid (alkyl)(mono)esters, the reaction may be repeated several times, for example by implementing several reactors in a cascade and intermediate draw-off systems.

Most preferably, the mixture resulting from the transesterification step comprises mono-, di- or triglyceride lower contents. The glycerides, as a whole, represent generally less than 3% by weight of the mixture total weight, preferably less than 1%.

The resulting lipid phase is then submitted to a concentration step c) so as to obtain a mixture enriched with the unsaponifiable fraction.

The preliminary concentration of oil to unsaponifiable enables to reduce the amount of engaged matter upon the possible subsequent step of saponification, and thus the amount to be extracted.

The concentration step c) may in particular be conducted by distillation or crystallization, especially cold crystallization or crystallization through evaporation under vacuum. As used herein, distillation is intended to mean any method known from the person skilled in the art especially, molecular distillation, distillation under atmospheric pressure or under vacuum, multi-stage, serially (especially in a wiped-film evaporator or a falling-film evaporator), azeotropic distillation, hydrodistillation, steam distillation, deodorization especially in thin-layer deodorizer under vacuum with or without steam injection or inert gas injection (nitrogen, carbon dioxide).

The most preferred method is the molecular distillation, which is intended to mean a fractional distillation under high vacuum and high temperature, but with a very short contact time, which prevents or limits the denaturation of heat-sensitive molecules.

This step of molecular distillation, as well as all other molecular distillations that can be carried out in the methods of the present invention, is conducted by using a short-path distillation unit, preferably a device chosen from molecular distillation devices of the centrifuge type and molecular devices of the wiped-film type.

Molecular distillation devices of the centrifuge type are known from the person skilled in the art. For example, the application EP-0 493 144 describes a molecular distillation device of this type. Generally speaking, the product to be distilled is spread in a thin layer on the heated surface (hot surface) of a conical rotor rotating at high speed. The distillation chamber is placed under vacuum. In these conditions, an evaporation of the unsaponifiable components occurs, not an ebullition, from the hot surface, the advantage being that delicate products are not degraded during evaporation.

Molecular distillation devices of the wiped-film type, also known from the person skilled in the art, comprise a distillation chamber provided with a rotating scraper, enabling the continuous spreading onto the evaporation surface (hot surface) of the product to be distilled. The vapors of product are condensed by means of a cold finger, placed in the middle of the distillation chamber. The external power and vacuum supply systems are very similar to those of a distillation unit of the centrifuge type (supply pumps, vacuum pumps with sliding vanes and oil diffusion, etc.). The recovery of residues and distillates in glass flasks occurs by gravitational flow.

The molecular distillation is conducted preferably at a temperature ranging from 100 to 260° C. by keeping a pressure ranging from 10⁻³ to 10⁻² mm Hg and preferably of about 10⁻³ mm Hg. The concentration of unsaponifiable matter in the distillate may reach 40% by weight. In the case of avocado, because of the very short contact time of the compounds with the heated area (a few milliseconds to one second), the cyclization of furan lipid precursors to furan lipids remains very limited at this stage.

Distillation generally enables to obtain a light fraction (first distillate), mainly comprising glycerides (mainly triglycerides) and, to a lesser extent, free fatty acids, natural and light paraffins, terpenes, and at least one heavier fraction (second distillate or residue), comprising the unsaponifiable fraction diluted in glycerides (mainly triglycerides). If a transesterification has been carried out, a light fraction will be obtained, which comprises fatty acid esters of high purity, and at least one heavier fraction comprising the unsaponifiable fraction diluted in residual fatty acid esters.

In the case of avocado, the concentrate enriched with the unsaponifiable fraction (and depleted in triglycerides or fatty acid esters, as the case may be) contains at this stage furan lipid precursors (that are weakly volatile).

The mixture enriched with the unsaponifiable fraction is thereafter submitted to a liquid-liquid extraction step d) in the presence of at least one polar organic solvent and at least one non-polar cosolvent immiscible with said polar organic solvent. Solvents and cosolvents can be used, that are anhydrous or not, and preferably solvents with a sufficiently low boiling point to allow distillation. This step is preferably carried out without any catalyst, in particular with no basic catalyst.

Step d) is generally conducted at room temperature, but may also be conducted by implementing a heating process at a temperature of at least 40° C., and preferably lower than or equal to 80° C., and more preferably lower than or equal to 75° C. In the case of avocado, step d) should be conducted at a temperature lower than or equal to 80° C., preferably lower than or equal to 75° C.

This step enables to isolate a fraction enriched with polar lipid components, functionalized especially by one or more hydroxyl, epoxide, ketone, thiol, aldehyde, ether or amine functions, whether unsaponifiable or not, as well as a fraction enriched in non-polar or weakly polar lipid components, especially components which do not contain (or just a few) hydroxyl, epoxide, ketone, thiol, aldehyde, ether and amine functions.

The use of two solvents during the liquid-liquid extraction causes the formation of a biphasic medium with two organic phases that are very different from each other as regards their composition. On one hand, lipid components, which are not (or not much) functionalized with one or more polar function(s) will be found preferably in the non-polar phase, whereas lipid components functionalized especially with one or more hydroxyl, epoxide, ketone, thiol, aldehyde, ether or amine function(s) will be found preferably in the polar phase.

This step enables the selective extraction of lipid components (unsaponifiable or not) functionalized especially with one or more hydroxyl, epoxide, ketone, thiol, aldehyde, ether or amine function(s), preferably several of them, and which are separated from the lipid component mixture (especially triglycerides or fatty acid esters, depending on the situation) not comprising such functions (or few), present in the medium at the end of the concentration step. Depending on the type of raw material used, these functionalized lipid components can be, without limitation, polyhydroxylated fatty alcohols and keto-hydroxylated compounds, that are furan lipid precursors (especially compound P1 H7 previously mentioned, precursor of linoleic furan H7) which are present in avocado, non esterified sterols, or esters of the following fatty acids: ricinoleic acid (12-hydroxy cis 9-octadecenoic acid) especially present in castor oil, lesquerolic acid (14-hydroxy-11-eicosanoic acid), densipolic acid (12-hydroxy-9,15-octadecadienoic acid) and auricolic acid (14-hydroxy-11,17-eicosadienoic acid), all three especially present in species of the Lesquerrella genus, coriolic acid (13-hydroxy-9,11-octadecadienoic acid), kamlolenic acid (18-hydroxy-9,11,13-octadecathenoic acid), especially present in oil extracted from seeds of the Kamala tree, coronaric acid (9,10-epoxi-cis-octadec-12-enoic) especially present in sunflower oil, vernolic acid (cis-12,13-epoxioleic acid) especially present in oil extracted from seeds of Euphorbia lagascae or from plants of the Vernonia genus.

The polar organic solvent may especially be a synthetic organic solvent chosen from light alcohols, ethers (in particular diethylether, diisopropyl ether, methyltertiobutyl ether, methyl tetrahydrofuran, 2-ethoxy-2-methylpropane), ketones (especially methyl isobutyl ketone, 2-heptanone), esters such as propionates (especially ethyl propionate, n-butyl propionate, isoamyl propionate), ketoalcohols such as diacetone alcohol, ether-alcohols such as 3-methoxy-3-methyl-1-butanol (MMB), phenols, amines, aldehydes, dimethyl formamide (DMF), dimethyl sulfoxide (DMSO), dimethyl isosorbide (DMI), water and combinations thereof.

The polar organic solvent preferably comprises at least one light alcohol. As used herein, a light alcohol is intended to mean an alcohol (comprising one or more hydroxyl function(s)), which molecular weight is lower than or equal to 150 g/mol, linear or branched, preferably C₁-C₆, more preferably, C₁-C₄. Preferably the light alcohol is a monoalcohol. It is preferably an aliphatic alcohol and most preferably an aliphatic monoalcohol, preferably chosen from methanol, ethanol, n-propanol, isopropanol, n-butanol, n-pentanol, n-hexanol, ethyl-2-hexanol, and isomers thereof.

The non-polar cosolvent, immiscible with the polar solvent (in the conditions of the liquid-liquid extraction), is preferably chosen so that lipid components, functionalized especially with one or more hydroxyl, epoxide, ketone, thiol, aldehyde, ether or amine function(s), to be extracted, are not soluble in this cosolvent. Considering their chemical nature, these functionalized lipid components will have necessarily a stronger affinity with the polar phase than with the non-polar solvent phase, in which they are not much (preferably not) soluble.

The non-polar cosolvent is an organic solvent which may especially be hexane, heptane, benzene, bicyclohexyl, cyclohexane, paraffin alkanes of vegetable origin obtained by dehydration of natural alcohols (or their Guerbet homologues) or by hydrotreatment of the lipids or biomasses (hydroliquefaction method) or by decarboxylation of the fatty acids, decaline, decane, kerosine, kerdane (a combustible hydrocarbon cut heavier than hexane), gas oil, lamp oil, methylcyclohexane, tetradecane, supercritical CO₂, pressurized propane or butane, natural non-polar solvents such as terpenes (limonene, alpha- and beta-pinene, etc.). It will preferably be an alkane or a mixture of alkanes, preferably hexane.

The preferred polar solvent/non-polar cosolvent couple is the methanol/hexane couple.

Moreover, water can be added to the binary mixture of solvents so as to extract especially more efficiently highly polar compounds, in particular hydroxylated compounds, wherein the amount of engaged water preferably represents from 0.1 to 20% by weight of the mixture of solvents, preferably from 0.5 to 5%.

To optimize the separation of the various lipid components between polar and non-polar phases, the extraction process may be repeated several times, for example by implementing several reactors in a cascade. Step d) may be in particular conducted in a co- or counter-current extraction column or by means of a battery of mixer-settlers, extraction columns or centrifugal extractors.

In order to be adapted to the industrial scale, a continuous extraction can be provided in a device for a continuous liquid-liquid extraction, such as in a pulsed column, a mixer-settler or equivalents. In a preferred embodiment the concentrate to be extracted and the solvent mixture (polar solvent and non-polar solvent) are introduced in counter-current to each other.

The (preferably alcoholic) polar phase, in which are especially soluble lipids functionalized with one or more function(s) chosen from hydroxyl, epoxide, ketone, thiol, aldehyde, ether and amine functions, such as polyhydroxylated fatty alcohols and furan lipid precursors (in the case of avocado), is separated from the non-polar phase. Said polar phase may further comprise, depending on the type of raw material used, triglycerides (or fatty acid esters, as the case may be), soluble polysaccharides, phenolic compounds, glucosinolates, isocyanates, polar alkaloids, polar terpenes.

The polar solvent (generally a light alcohol) is evaporated from the polar phase in particular under reduced pressure, optionally by implementing a heating process. In the case of avocado, if the evaporation temperature is high (especially of about 80° C. or higher), a cyclization of the furan lipid precursors to furan lipids may already occur at this early stage. The lipid product obtained may be submitted to a step of decantation or centrifugation which enables to separate the residual soaps from water, and/or to a filtration and/or washing step. The remaining lipid phase may then be washed with water and dried under vacuum.

In order to be positively reused, the non-polar solvent phase may be submitted to a solvent evaporation step conducted under vacuum and at a suitable temperature. The vaporized solvent is then condensed for being recycled. The mixture mainly composed of glycerides and non-polar unsaponifiable (or not) compounds may then be engaged in a transesterification step, then in a molecular distillation so as to obtain, on one hand, purified esters (in the distillate) and, on the other hand, a distillation residue enriched with non-polar minor compounds. The extraction of these essentially unsaponifiable compounds is conducted according to methods that are known to the person skilled in the art. For example, by conducting the following sequence: 1) saponification of the alkyl esters, 2) liquid-liquid extraction enabling to separate the unsaponifiable compounds from the soaps, 3) removing the solvent of the solvent phase enriched with unsaponifiable matters and 4) final purification of the unsaponifiable matter.

The resulting polar lipid phase (mainly composed of glycerides or fatty acid esters, as the case may be, optionally of free fatty acids, and enriched with polar unsaponifiable compounds) is then optionally submitted to a heat treatment step at a temperature higher than or equal to 75° C., preferably higher than or equal to 80° C.

In the case of avocado, the heat treatment step at 75-80° C., or above, of the lipid phase is compulsory. It is intended to make the cyclization of the furan lipid precursors to furan lipids effective. This step may be conducted before, after or during the saponification step (if any), preferably before, because saponification would otherwise convert the furan lipid precursors to modified unsaponifiable derivatives (that is to say different from the furanic compounds), which would be less interesting. The duration of such treatment generally ranges from 0.5 to 5 hours, depending on the heating method used. The temperature set for the treatment is generally lower than or equal to 150° C., preferably lower than or equal to 120° C. It should be naturally understood that temperature and reaction time are two parameters that strongly depends from each other as regards the expected result of the heat treatment, which consists in promoting the cyclization of the furan lipid precursors.

Advantageously, this heat treatment is carried out under inert atmosphere, especially under a nitrogen continuous flow. It is preferably conducted under atmospheric pressure.

The heat treatment step may be implemented in the presence, or not, of an acid catalyst. As used herein, an acid catalyst is intended to mean mineral and organic catalysts, said to be homogeneous, such as hydrochloric, sulfuric, acetic or paratoluenesulfonic acids, but also, and preferably, heterogeneous solid catalysts, such as silica, alumina, silica-alumina, zirconias, zeolites, acidic resins. Acidic aluminas with high specific areas will be in particular selected, that is to say at least equal to 200 m²/g. Preferred for implementation of the method of the invention are catalysts of the acidic alumina type.

The resulting lipid phase having optionally undergone the heat treatment may then be submitted to steps of e) saponification and f) extraction of the unsaponifiable fraction from the saponified mixture, depending on the type of raw material used. In the case of avocado, especially, steps e) and f) are performed, so as to separate glycerides (or fatty acid esters, as the case may be). In other cases, steps e) and f) can be omitted and an oil can be isolated, containing the unsaponifiable fraction, together with other compounds, such as glycerides (or fatty acid esters, if a transesterification process was effected), especially triglycerides. If no transesterification occurred, this oil may in particular comprise polar compounds, saponifiable or not, that are sensitive in a basic medium.

Saponification is a chemical reaction, which converts an ester to a water-soluble carboxylate ion and to alcohol. In the present case, saponification especially transforms fatty acid esters (for example triglycerides) to fatty acids and to alcohol, the released alcohol being primarily glycerol, or the light alcohol if a transesterification was carried out.

The saponification step may be implemented in the presence of potash or soda in an alcoholic medium, preferably ethanol. Typical experimental conditions include a reaction in the presence of potash 12N under reflux of ethanol for 4 hours. At this stage, and optionally, a cosolvent may be advantageously used so as to improve in particular the reaction kinetics or to protect unsaponifiable compounds sensitive to basic pH values. This cosolvent may especially be chosen from terpenes (limonene, alpha- and beta-pinene, etc.), alkanes, especially paraffins.

General publications such as Bailey's Industrial Oil and Fat Products, 6^{th} Edition (2005), Fereidoon Shahidi Ed., John Wiley & Sons, Inc., and March's Advanced Organic Chemistry: Reactions, Mechanisms, and Structure, 5^{th} Edition (2001), M. B. Smith, J. March, Wiley-Interscience, describe in more details the conditions of the saponification step, as well as of the optional transesterification step.

Thereafter the unsaponifiable fraction is one or more times extracted from the saponified mixture. This step is preferably performed by liquid-liquid extraction by means of at least one suitable organic solvent, that is to say, which is immiscible with the alcoholic or hydroalcoholic solution resulting from the saponification. It enables to separate the fatty acid salts (soaps) formed during the saponification process of the unsaponifiable fraction.

The organic solvent may especially be a synthetic organic solvent chosen from optionally halogenated alkanes (especially petroleum ether or dichloromethane), aromatic solvents (especially trifluorotoluene, hexafluorobenzene), halogeno-alkanes, ethers (especially diethyl ether, diisopropyl ether, methyltertiobutyl ether, methyl tetrahydrofuran, 2-ethoxy-2-methylpropane), ketones (especially methyl isobutyl ketone, 2-heptanone), propionates (especially ethyl propionate, n-butyl propionate, isoamyl propionate), hexamethyldisiloxane, tetramethylsilane, diacetone alcohol, 1-butoxymethoxy butane, 3-methoxy-3-methyl-1-butanol (MMB), or a natural organic solvent chosen from terpenes, such as limonene, alpha pinene, beta pinene, myrcene, linalol, citronellol, geraniol, menthol, citral, citronellol, or oxygenated organic derivatives of natural origin, especially ethers, aldehydes, alcohols and esters, such as for example furfural and furfurol. A terpene will be preferably chosen. The extraction may be conducted in a co- or counter-current extraction column or by means of a battery of mixer-settlers, extraction columns or centrifugal extractors.

In order to be adapted to the industrial scale, a continuous extraction can be provided in a device for a continuous liquid-liquid extraction, such as in a pulsed column, a mixer-settler or equivalents.

Once extracted, the unsaponifiable fraction is preferably purified, in particular by decantation and/or centrifugation (glycerol removal in the case of triglyceride saponification), solvent removal, washing, drying, filtration and/or deodorization under vacuum. More precisely, the purification step may especially be conducted by implementing one or more of the following sub-steps:

centrifugation of the solvent phase so as to extract the residual soaps, then filtration,

washing, with water optionally saturated with sodium chloride, of the solvent phase, in order to remove the alkaline residual traces,

drying through evaporation of the extraction solvent through distillation under vacuum, hydrodistillation or azeotropic distillation,

deodorization under vacuum of the unsaponifiable fraction so as to extract therefrom, in the deodorization conditions, any remaining contaminant especially the extraction solvent, pesticides, polycyclic aromatic hydrocarbons.

The first method of the invention enables to obtain a high-purity unsaponifiable fraction enriched with polar compounds (except, this is particular, in the case of avocado, furan lipids, which due to their weakly polar nature, are present in the unsaponifiable fraction isolated with the first method of the invention, because they have been formed in situ from polar precursors after a selective extraction step of the polar compounds). In a non-exhaustive manner, the unsaponifiable compounds obtained at the end of the implementation of the present method in the fraction isolated in fine may be, depending on the nature of the raw material used, optionally polyhydroxylated fatty alcohols, furan lipids (in the case of avocado), non-esterified (free) or non-glycosylated sterols and triterpene alcohols, free and glycosylated polyphenols, free or sulfated cholesterol, lignanes, phorbol esters, triterpenic acids (for ex. ursolic acid), polar terpenes (mono-, di- and sesqui-terpenes, with an alcohol function), alkaloids, polycosanols, limonoids, xanthophylls (lutein, astaxanthin, zeaxanthin) in a free form, gossypol, karanjin, shizandrin, azadirachtin, co-enzyme Q10, aflatoxins, especially B1 and B2, isoflavones, caffeine, theobromine, yohimbine, sylimarin, lupeol, allantoin.

In a general way, the average composition of an avocado unsaponifiable obtained following these different steps (amongst which steps e) and f)) as expressed in percentages by weight compared to the unsaponifiable total weight is as follows:

furan lipids 50-75%

polyhydroxylated fatty alcohols 5-30%

squalene 0.1-5%

sterols 0.1-5%

others 0-15%

According to the present invention, the unsaponifiable matter obtained as described may then be submitted to a (second) step of distillation, so as to further improve the purity thereof, preferably a molecular distillation, conducted preferably at a temperature ranging from 100 to 160° C., more preferably from 100 to 140° C., under a pressure ranging preferably from 10⁻³ to 5.10⁻² mm Hg. According to another embodiment, the set temperature varies from 130 to 160° C.

The temperature and pressure chosen for this distillation influence the formation of the recovered distillate. Thus, this (second) distillation may enable to obtain a distillate comprising primarily, in the case of avocado, avocado furan lipids, the purity of which may be higher than 90% by weight, when the distillation temperature varies from 100 to 140° C. When the distillation temperature varies from 130 to 160° C., a distillate is generally obtained comprising primarily avocado furan lipids and to a lesser extent polyhydroxylated fatty alcohols from avocado, which combined amounts may exceed 90% by weight.

This first method of the invention enables thus to provide a selective extraction not only of the avocado furan lipids, but also of avocado polyhydroxylated fatty alcohols, if desired.

Furthermore, the unsaponifiable compounds obtained at the end of the implementation of the method in the fraction isolated from the non-polar solvent phase, may be in fine, depending on the nature of the raw material used, sterol esters, esterified triterpene alcohols, cholesterol esters, tocopherols (and corresponding tocotrienols), sesamolin, sesamin, sterenes, squalene, paraffin hydrocarbons, weakly to non-polar terpenes (mono-, di- and sesqui-terpenes with an aldehyde and/or a ketone function), esterified xanthophylls (lutein, astaxanthin, zeaxanthin), carotenoid type pigments (beta-carotene, lycopene), waxes, calciferol, cholecalciferol, pongamol.

The second method of the invention will now be presented by explaining essentially the differences as compared to the first method of the invention. It should be noted that the description of the first method of the invention can be referred to, as regards all other characteristics, which are common to both methods.

The renewable raw materials used in the second method of the invention are not particularly limited and optionally comprise lipid components functionalized with one or more hydroxyl, epoxide, ketone, thiol, aldehyde, ether or amine function(s). They comprise necessarily those lipid components, which are not functionalized by any of the previously mentioned functions (or by a few number of these functions), these components being the most commonly encountered in nature.

This method optionally comprises a first step a) of dehydration and/or of conditioning of the renewable raw material. Dehydration and conditioning are not necessarily conducted at a temperature lower than or equal to 80° C. or 75° C. Said temperature is preferably higher than or equal to −50° C. When a heating process is provided, the temperature generally varies from 50 to 120° C., more preferably from 75 to 120° C.

As for the first method, dehydration may be implemented before or after conditioning (if any). It lasts preferably from 8 to 36 hours.

The renewable raw material optionally undergoes (this is the case for avocado in particular) a heat treatment as described especially in the French patent application FR 2678632, at a temperature higher than or equal to 75° C., preferably higher than or equal to 80° C., before step d) of liquid-liquid extraction, which will be described hereafter. Most preferably, the heat treatment and the dehydration of the raw material, if both apply, occur simultaneously and form a single step.

In the case of avocado, this heat treatment step at 75° C. or above of the raw material having been beforehand, or not, conditioned and/or dehydrated, is compulsory. As for the first method described, it is intended to promote the cyclization of the furan lipid precursors to furan lipids. The duration of such treatment generally varies from 8 to 36 hours, depending on the heating method used. The temperature set for the treatment is generally lower than or equal to 150° C., preferably lower than or equal to 120° C. Advantageously, such a heat treatment is conducted under inert atmosphere, especially under a nitrogen continuous flow. It is preferably conducted under atmospheric pressure.

Once optionally dehydrated and optionally conditioned, the raw material undergoes a step b) of extraction of the fats therefrom resulting in the production of an oil. This is preferably effected with no catalyst, in particular with no basic catalyst.

Step b) is not necessarily conducted at a temperature lower than or equal to 80° C. or 75° C. It may be effected without limitation as regards temperature and whatever the treated raw material and may exceed 75 or 80° C. Step b) is generally conducted at room temperature, but may also be conducted by implementing a heating process at a temperature ranging from 40 to 100° C., preferably lower than or equal to 80° C., more preferably lower than or equal to 75° C.

As for the first method, an oil is extracted from the solid raw material, optionally using a solvent. In this case, the solvent may be evaporated in particular under reduced pressure, without special precautions as regards the heating process optionally used so as to evaporate the solvent, since the conversion of the furanic lipid precursors to furanic lipids does not need to be particularly avoided.

The resulting lipid phase may optionally be submitted to a transesterification step in the presence of at least one polar organic solvent comprising at least one light alcohol, such as previously defined, and at least one catalyst, before or after the concentration step c), preferably before. In any event, the transesterification must be carried out before step e) of saponification.

The resulting lipid phase is then submitted to a concentration step c) so as to obtain a mixture enriched with the unsaponifiable fraction.

The concentration may be implemented before or after the heat treatment, if any, or these two steps may be conducted concomitantly, if the concentration requires a heating process at a suitable temperature. The concentration is preferably carried out prior to effecting the heat treatment, in particular in the case of avocado.

As for the first method, the preferred concentration method is the molecular distillation. It is also possible to perform a classical distillation, which, In the case of avocado, would simultaneously enable upon concentration the complete cyclization of the furan lipid precursors (if not already effected) through a heating process at 75° C. or above, preferably at 80° C. or above.

Distillation generally enables to obtain a light fraction (first distillate), comprising primarily glycerides (mainly triglycerides) and, to a lesser extent, free fatty acids, natural and light paraffins, terpenes, and at least one heavier fraction (second distillate or residue), comprising the unsaponifiable fraction diluted in glycerides (mainly triglycerides). If a transesterification has been carried out, a light fraction will be obtained, which comprises fatty acid esters of high purity, and at least one heavier fraction comprising the unsaponifiable fraction diluted in residual fatty acid esters.

In the case of avocado, if no transesterification was carried out, and if the heat treatment step at a temperature higher than or equal to 75° C. or 80° C. was conducted before the concentration step c), or occurred during this step, a concentrate is isolated, enriched with the unsaponifiable fraction (and depleted in triglycerides) and containing at this stage furan lipids (that are more volatile than triglycerides), typically in an amount of about 10 to 15% by weight. If said heat treatment is effected after step c) or is completed after step c), a concentrate is isolated, enriched in unsaponifiable fraction (and depleted in triglycerides), containing at this stage furan lipid precursors and possibly already formed furan lipids.

In the case especially of avocado, the heat treatment at temperature higher than or equal to 75° C., preferably higher than or equal to 80° C., is effected before the liquid-liquid extraction step d), in particular after step c), before step c), during step c) or during step a). Several partial heat treatments conducted before step d) may also lead to a complete heat treatment resulting in the total conversion of the furan lipid precursors to furan lipids.

The mixture enriched with the unsaponifiable fraction is then submitted to a liquid-liquid extraction step d) in the presence of at least one polar organic solvent and at least one non-polar cosolvent immiscible with said polar organic solvent. As in the first method, these solvents and cosolvents may be anhydrous or not, and water may be added to the extraction solvent mixture.

Step d) is generally conducted at room temperature but may also be conducted by implementing a heating process, with no limitation as regards the temperature (as opposed to that of the first method), where said temperature may vary from 40 to 100° C., as in the first method.

This step enables to isolate an organic fraction enriched with non-polar (or weakly polar) lipid components, that is to say not containing any (or not much) hydroxyl, epoxide, ketone, thiol, aldehyde, ether and amine function, whether unsaponifiable or not, as well as a fraction enriched with polar lipid components, especially components functionalized with or more of hydroxyl, epoxide, ketone, thiol, aldehyde, ether and amine function(s).

This step essentially enables to set the lipid components apart, which comprise one or more of these functions, preferably many of them (for example polyols),

Depending on the type of raw material used, these lipid components not or only weakly polar that have been isolated during step d), may be, without limitation, glycerides (or fatty acid esters resulting from the transesterification, as the case may be) not containing any of hydroxyl, epoxide, ketone, thiol, aldehyde, ether and amine functions, furan lipids (in the case of avocado, furan lipid precursors have already been converted to furan lipids prior to beginning the liquid-liquid extraction step, these furan lipids being non hydroxylated), weakly polar alcohols, such as tocopherols, squalene, xanthophylls and esterified sterols.

The non-polar cosolvent, immiscible with the polar solvent (in the conditions of the liquid-liquid extraction), is preferably chosen so that lipid components, functionalized especially with one or more hydroxyl, epoxide, ketone, thiol, aldehyde, ether or amine function(s) and to be not extracted, are not soluble in such cosolvent. Considering their chemical nature, these functionalized lipid components will have necessarily a stronger affinity with the polar phase than with the non-polar solvent phase in which they are not much (preferably not) soluble.

The non-polar cosolvent is evaporated from the non-polar phase enriched with lipids not containing any of the hydroxyl, epoxide, ketone, thiol, aldehyde, ether and amine functions (or few of them) (unsaponifiable or not) especially under reduced pressure. The lipid product obtained may be submitted to a step of neutralization (before or after the evaporation of the non-polar cosolvent, preferably before), preferably through an acid, then to a step of decantation or centrifugation, and/or to a step of filtration. The remaining lipid phase may then be washed with water and dried under vacuum.

The resulting lipid phase (phase typically composed essentially of glycerides or fatty acid esters resulting from the transesterification, as the case may be, possibly of free fatty acids and enriched with non-polar unsaponifiable compounds) is then optionally submitted to steps e) of saponification and f) of extraction of the unsaponifiable fraction from the saponified mixture. Once extracted, the unsaponifiable fraction is preferably purified, using the same procedures as described in the first method of the invention.

The second method according to this invention enables to obtain a very pure unsaponifiable fraction, enriched with weakly polar to non-polar compounds. In a non-exhaustive manner, the unsaponifiable compounds obtained at the end of the implementation of such method in the fraction isolated in fine may be, depending on the nature of the raw material used, furan lipids (in the case of avocado), sterol esters, esterified triterpene alcohols, cholesterol esters, tocopherols (and corresponding tocotrienols), sesamolin, sesamin, sterenes, squalene, paraffin hydrocarbons, weakly to non-polar terpenes (mono-, di- and sesqui-terpenes with an aldehyde and/or a ketone function), esterified xanthophylls (lutein, astaxanthin, zeaxanthin), carotenoid type pigments (beta-carotene, lycopene), waxes, calciferol, cholecalciferol, pongamol.

In a general way, the average composition of an avocado unsaponifiable obtained following these different steps (amongst which steps e) and f)), as expressed in percentages by weight compared to the unsaponifiable total weight, is given thereunder:

furan lipids 60-80%

squalene 1-7%

others 5-20% (hydrocarbons, tocopherols, fatty ketones, heavy pigments . . . )

polyhydroxylated fatty alcohols 0.1-10%.

According to the present invention, the unsaponifiable matter obtained as described may then be submitted to a (second) step of distillation, so as to further improve the purity thereof, preferably a molecular distillation, conducted preferably at a temperature ranging from 100 to 160° C., more preferably from 100 to 140° C., under a pressure ranging preferably from 10⁻³ to 5.10⁻² mm Hg. This (second) distillation may enable to obtain a distillate comprising primarily, in the case of avocado, avocado furan lipids, the purity of which may be higher than 90% by weight.

This second method of the invention thus enables to obtain a selective extraction of avocado furan lipids, except the polyhydroxylated fatty alcohols from avocado which have been extracted in the polar phase during the liquid-liquid extraction step.

Furthermore, the unsaponifiable compounds obtained at the end of the implementation of such method in the fraction isolated from the polar solvent phase, in fine may be, depending on the nature of the raw material used, the optionally polyhydroxylated fatty alcohols, furan lipids (in the case of avocado), non-esterified (free) or non-glycosylated triterpene alcohols and sterols, free and glycosylated polyphenols, free or sulfated cholesterol, lignanes, phorbol esters, triterpene acids (for ex. ursolic acid), polar terpenes (mono-, di- and sesqui-terpenes, with an alcohol function), alkaloids, polycosanols, limonoids, xanthophylls (lutein, astaxanthin, zeaxanthin) in a free form, gossypol, karanjin, shizandrin, azadirachtin, co-enzyme Q10, aflatoxins, especially B1 and B2, isoflavones, caffeine, theobromine, yohimbine, sylimarin, lupeol, althetoin.

The present invention has many advantages as compared to traditional existing methods used for the extraction from oils or deodorization emissions. First of all, the method of the invention is economical because it does not require the substantial investments of the traditional methods. As regards investment, the method of the invention enables to avoid the use of refining tools (mucilage removal, neutralization).

In addition, the present invention is very interesting as regards co-utilization, because implementing the methods of the invention leads to high-added value co-products, such as:

oil cakes, from which toxic or antinutritional compounds optionally present in the initial biomass have been removed, and which are directly utilizable in animal feeding or human nutrition, or oil cakes, sources of interesting oligopeptides and/or oligosaccharides,

polysaccharides and polyphenols utilizable in cosmetics, pharmacy and animal feeding and human nutrition.

From an economic and environmental point of view, the methods of the invention not only enable to reuse almost 100% of the fruit, as opposed to current methods and therefore to save biomass, or even cultivated areas, but they also enable to improve the whole value chain, from the farmer upstream to the user downstream, of said unsaponifiable matters. Lastly, they respect the key-principles of today's biorefinery models that are being developed for many applications, in particular for energetic and industrial purposes.

The unsaponifiable fractions obtained by the methods of the invention share a composition close or even similar to that of the unsaponifiable present in the raw material before the treatment.

Advantageously, these unsaponifiable fractions and these co-products of the invention are devoid of any residual toxic solvent and thus have a much better regulatory safety and acceptability as compared with products resulting from traditional methods. These particular characteristics enable a more adapted use of the unsaponifiable fractions obtained by the methods of the invention and/or of the co-products provided, in cosmetic, drug, food compositions or food supplements or additives for humans and/or animals.

Likewise, the method of the invention will enable to separate and/or concentrate, depending on their polarity, the contaminants that may be present in vegetable or animal biomasses: polycyclic aromatic hydrocarbons (PAHs), pesticides, polychlorobiphenyls (PCB), dioxins, brominated flame retardants, pharmaceuticals, etc.

The avocado unsaponifiable fraction obtained by the methods of the invention may especially be used for preparing a drug for the treatment, for example, of joint affections, more particularly the treatment of osteoarthritis and for the treatment of arthritis (that is to say rheumatoid arthritis, psoriatic arthritis, Lyme disease and/or any other type of arthritis). The thus prepared drug may be intended for the treatment of periodontal diseases, and in particular for the treatment of periodontitis. This drug may furthermore be suitable for treating osteoporosis. Moreover, this drug may be intended to modulate the nervous cell differentiation induced by NGF (Nerve Growth Factor). Lastly, this drug may be intended to repair tissues, and in particular the skin tissues, especially in the frame of a dermatological application.

The avocado unsaponifiable fraction derived from the methods of the invention may also be employed in cosmetic compositions, especially in dermocosmetics, for the cosmetic treatment of skin, adjacent mucosae and/or keratinized skin appendages (aging, scars . . . ), of hair fibers or dermal papillae, in the presence of an excipient and/or a cosmetically acceptable vehicle.

Likewise, the co-products of the method, such as proteins and carbon hydrates, may, depending on their nature, lead as such or post transformation, to the production of active principles or excipients for use in pharmacy, cosmetics and human nutrition or animal feeding applications.

### EXAMPLE

Selective Extraction of Unsaponifiable Compounds from Avocado

20 kg of whole Fuerte avocados are cut (stone included) in maximum 0.5 cm-thick slices. 19 kg of such slices are then dried in a ventilated drying oven at 90° C. for 16 hours (batch A). As a result, 2649 g of dried avocado are obtained after drying. The residual moisture determined by thermogravimetry at 105° C. is of 6.3%.

The amount of lipids in homogenate A is then determined according to a standardized method (NF EN ISO 659): 47.3% by weight of dry matter.

Batch A is then submitted to following actions:

1) coarse powder grinding (particle size ranging from 0.3 to 0.8 cm diameter);

2) introduction of the homogenate (2000 g) into a packed-bed percolation column;

3) 2000 g of high-purity hexane (>99%) are then sent to the flake bed for 30 minutes at 40° C. of a thermoregulated percolation column;

4) the miscella (solvent phase resulting from the liquid-solid extraction) is then racked off. The flake bed is then washed through 5 successive washing operations with hexane at 40° C. (5 minutes per washing, 1000 g of hexane per washing);

5) the whole miscella is then gathered, and filtered on a Büchner filter. The filtered hexane phase is then distilled on a rotary evaporator under a 20 mBar vacuum at 90° C. for 20 minutes. 912.9 g of avocado oil are collected.

6) The oil obtained is then distilled under a 10⁻³ mm Hg vacuum at 230° C. in a wiped-film distillator (supply rate 2.3 kg/h). 47.6 g of distillate are obtained. The amount of unsaponifiable matter in the distillate, such as determined by the NF ISO 3596 standardized method as modified, wherein the extraction solvent is dichloroethane: 23.7% by weight.

7) 30 g of the distillate are then mixed with 30 g of hexane and 30 g of ethanol and 0.5 g of demineralized water in a funnel. After stirring and decantation of the medium, a 2-phase mixture is obtained.

The heavy phase (ethanol) is then recovered in a funnel and extracted three times using a mixture composed of 15 g of hexane, 15 g of ethanol and 0.25 of water. The phases, on one hand the hexane and on the other hand the ethanol phases, are gathered, then evaporated separately on a rotary evaporator (20 mbar vacuum, temperature 90° C. for 20 minutes). From the organic phases are obtained 23.2 g of an oil derived from the hexane phases and 5.6 g of an oil derived from the ethanol phases. The contents in unsaponifiable matter in these two oils are determined according to the standardized method NF ISO 3596 as modified (extraction solvent dichloroethane):
14.9% by weight for the oil derived from the hexane phases
16.33% by weight for the oil derived from the ethanol phases.

A thin-layer chromatography analysis (TLC) indicates that the lipids derived from the hexane phases comprise high amounts of furan compounds with some traces of avocado polyhydroxylated fatty alcohols, where these compounds reveal TLC specific spots. Likewise, the analysis of the lipids derived from the ethanol phases comprises high amounts of avocado polyhydroxylated fatty alcohols and minor amounts (traces) of furan compounds.

As a consequence, the method indeed leads on one hand to the formation of lipids enriched with avocado polar unsaponifiable compounds (polyhydroxylated fatty alcohols), and on the other hand to lipids enriched with avocado non-polar unsaponifiable compounds (furan compounds).

1-11. (canceled)

12. A method for extracting an unsaponifiable fraction from a solid renewable raw material comprising fats, and in particular lipids functionalized with one or more function(s) chosen from hydroxyl, epoxide, ketone, thiol, aldehyde, ether and amine functions, comprising the following steps:
a) optional dehydration possibly preceded or followed with a conditioning of the renewable raw material,
b) extraction of the fats from the raw material optionally dehydrated and optionally conditioned to obtain an oil,
c) concentration of the oil resulting from step b) so as to obtain a mixture enriched in unsaponifiable fraction,
d) liquid-liquid extraction of the mixture enriched in unsaponifiable fraction in the presence of at least one polar organic solvent and at least one non-polar cosolvent immiscible with said polar organic solvent, leading to the formation of an organic polar phase enriched in lipids functionalized with one or more function(s) chosen from hydroxyl, epoxide, ketone, thiol, aldehyde, ether and amine functions,
and optionally comprising the following steps:
e) saponification of the polar organic phase, optionally preceded, accompanied or followed with a heat treatment at a temperature higher than or equal to 75° C., preferably higher than or equal to 80° C.,
f) extraction of the unsaponifiable fraction from the saponified mixture.

13. A method for extracting an unsaponifiable fraction from a solid renewable raw material comprising the following steps:
a) optional dehydration possibly preceded or followed with a conditioning of the renewable raw material,
b) extraction of the fats from the raw material optionally dehydrated and optionally conditioned to obtain an oil,
c) concentration of the oil resulting from step b) so as to obtain a mixture enriched in unsaponifiable fraction,
d) liquid-liquid extraction of the mixture enriched in unsaponifiable fraction in the presence of at least one polar organic solvent and at least one non-polar cosolvent immiscible with said polar organic solvent, leading to the formation of a non-polar organic phase enriched in lipids with no or few hydroxyl, epoxide, ketone, thiol, aldehyde, ether and amine functions, and optionally comprising the following steps:
e) saponification of the non-polar organic phase,
f) extraction of the unsaponifiable fraction from the saponified mixture,
wherein said renewable raw material undergoes optionally a heat treatment at a temperature higher than or equal to 75° C., preferably higher than or equal to 80° C., before step d).

14. The method according to claim 13, wherein step a) of dehydration is carried out, and said heat treatment is conducted concomitantly to step a) of dehydration.

15. The method according to claim 12, wherein the renewable raw material is chosen from the fruit, the stone, the leaves of avocado and their mixtures, said heat treatment is carried out, and steps a), b) and d) are carried out at a temperature lower than or equal to 80° C., preferably lower than or equal to 75° C.

16. A method according to claim 13, wherein the renewable raw material is chosen from the fruit, the stone, the leaves of avocado and their mixtures, and said heat treatment is carried out.

17. The method according to claim 12, wherein step a) of dehydration is carried out and dehydration is conducted so as to reach a residual moisture lower than or equal to 10% by weight, as compared to the weight of the raw material obtained at the end of the dehydration step.

18. The method according to claim 12, wherein the polar organic solvent is a light alcohol chosen from methanol, ethanol, propanol, isopropanol, butanol, pentanol, hexanol, ethyl-2-hexanol, and isomers thereof.

19. A method according to claim 12, wherein the non-polar cosolvent is an alkane or a mixture of alkanes.

20. The method according to claim 12, wherein steps b) and d) of extraction are carried out with no catalyst.

21. The method according to claim 12, wherein the oil concentration is carried out by molecular distillation.

22. The method according to claim 12, wherein the method comprises steps e) and f), the extraction of the unsaponifiable fraction from the saponified mixture being performed by liquid-liquid extraction using at least one organic solvent.

23. The method according to claim 13, wherein step a) of dehydration is carried out and dehydration is conducted so as to reach a residual moisture lower than or equal to 10% by weight, as compared to the weight of the raw material obtained at the end of the dehydration step.

24. The method according to claim 13, wherein the polar organic solvent is a light alcohol chosen from methanol, ethanol, propanol, isopropanol, butanol, pentanol, hexanol, ethyl-2-hexanol, and isomers thereof.

25. A method according to claim 13, wherein the non-polar cosolvent is an alkane or a mixture of alkanes.

26. The method according to claim 13, wherein steps b) and d) of extraction are carried out with no catalyst.

27. The method according to claim 13, wherein the oil concentration is carried out by molecular distillation.

28. The method according to claim 13, wherein the method comprises steps e) and f), the extraction of the unsaponifiable fraction from the saponified mixture being performed by liquid-liquid extraction using at least one organic solvent.
